(19) United States
(12) Patent Application Publication
(10) Pub. No.: US 2013/0281796 A1
(43) Pub. Date: Oct. 24, 2013

(54) BIOSENSOR WITH EXERCISE AMOUNT MEASURING FUNCTION AND REMOTE MEDICAL SYSTEM THEREOF

(71) Applicant: Lung-Te PAN, New Taipei City (TW)

(72) Inventor: Lung-Te PAN, New Taipei City (TW)

(73) Assignee: BroadMaster Biotech Corp.

(21) Appl. No.: 13/452,228

(22) Filed: Apr. 20, 2012

(51) Int. Cl. A61B 5/11; A61B 5/00

(52) U.S. Cl. 600/301

(57) ABSTRACT

A biosensor is provided with an exercise amount measuring function, having: a microprocessor, for processing signals and data; a movement-sensing module connected to the microprocessor, for detecting a movement signal of the user; a biological signal-detecting module connected to the microprocessor, for detecting a biological signal of the user; a flash memory connected to the microprocessor, for storing an exercise amount and biological signal data generated by the microprocessor; and a display unit connected to the microprocessor, for displaying the exercise amount and the biological signal data of the user.

### BACKGROUND OF THE INVENTION

1. Technical Field

The present invention relates to biosensors, and more particularly, to a biosensor with exercise amount measuring function.

2. Description of Related Art

With the rapid economic development and the change of modern people's lifestyle and dietary patterns, the global prevalence of diabetes has dramatically increased and diabetes has posed as one of the top challenges to the medical science. Diabetes is known as a chronic disease that cannot be cured completely but can only be effectively controlled to prevent complex complications caused thereby. Thus, for achieving good control of diabetes, a diabetic is always required to monitor his or her blood sugar level regularly. Currently, using biosensors to measure blood sugar level is one of the most popular ways for diabetics to record and thereby monitor their blood sugar levels every day. As such biosensors, many glucose meters, for example, measure blood sugar levels by way of electrochemical method in recent years. The electrochemical method involves using electrodes and immobilized glucose oxidase or glucose dehydrogenase to measure the blood sugar level of the diabetic.

It is well known that regular physical exercise is helpful to enhance reactions of various human cells to insulin, in turn facilitating blood glucose control. For patients who need insulin injection, taking regular physical exercise may allow them to reduce required dosage of insulin. As to patients who take oral antidiabetic agents, regular physical exercise may also help them to reduce dosage or even have their conditions suppressed only through diet control. However, when exercising, diabetics, especially those take antidiabetic agents or insulin injection, tend to have their blood glucose levels over low because skeletal muscles consume lots of energy. On the other hand, when excising with over high blood glucose levels, diabetics can have ketone bodies generated and causing dehydration. For these reasons, diabetics are usually recommended not to exercise when having a blood glucose level higher than 250 mg/dl or lower than 80 mg/dl. Therefore, diabetics have to carefully plan their exercise programs by considering their physical performance status and consulting medical staff. When exercising, they also have to carefully follow the programs, record calories burned, and watch blood glucose changes before and after exercise. In addition, diabetics are recommended to exercise regularly, at least once per day or once another day, for better control of their blood glucose levels. Hence, it is preferable that diabetics carry relevant measuring devices to measure and record calories burned during exercise, for them to plan and modify exercise programs accordingly.

Nevertheless, the existing exercise amount measuring devices, such as pedometers or wrist-wearing exercise amount measuring devices, are usually designed for measuring an established exercise amount and displaying only the calories burned, but incapable of indicating real-time blood glucose levels. Thus, diabetics have to additionally carry a biosensor when going out for exercise or have to go immediately to a place having blood glucose measuring equipment for measuring their blood glucose level after exercise. Thus, how to overcome the foregoing inconveniences would be an issue to address.

### BRIEF SUMMARY OF THE INVENTION

In view of the above mentioned drawbacks, the present inventor provides a biosensor with exercise amount measuring function. This inventive device combines the features of exercise amount measuring function and biological signal-sensing function, for not only displaying a real-time blood glucose level of a user, but also recording the user's daily exercise amount throughout the user's daily activities and converting the exercise amount into values of daily calories burned, so that the user is conveniently informed of his/her blood glucose changes and calories burned daily, for him/her to evaluate the impact of his/her current exercise level on the blood glucose level. Also, medical staff may take this information as a reference when planning an individual exercise program for the user or considering medical care for the user's diabetes.

Therefore, one aspect of the invention is to provide a biosensor with exercise amount measuring function, comprising: a microprocessor, for processing signals and data; a movement-sensing module connected to the microprocessor, for detecting a movement signal of the user; a biological signal-detecting module connected to the microprocessor, for detecting a biological signal of the user; a flash memory connected to the microprocessor, for storing an exercise amount and biological signal data generated by the microprocessor; and a display unit connected to the microprocessor, for displaying the exercise amount and the biological signal data of the user.

Another aspect of the invention is to provide a method for measuring an exercise amount using the biosensor of the present invention, comprising: an initialization step, where normal information is inputted; a personalization step, where personal information is inputted; a zeroing step, where an exercise amount is counted from zero; an accelerator-signal recording step, where a movement signal is recorded; a time determining step, where whether it is after 24:00 of a day is determined, and if it is not, the method returns to the accelerator-signal recording step; and if it is, an exercise amount summing step for calculating an accumulated exercise amount of the previous day is performed, and then the method returns to the zeroing step to count the exercise amount of a new day from zero.

A further aspect of the invention is to provide a remote medical system, comprising a network system, a home healthcare system connected to the network system and a medical care system connected to the network system, wherein the home healthcare system uses a biosensor of the present invention to sense movement signals and physiological signals from a user.

### BRIEF DESCRIPTION OF THE DRAWINGS

FIG. 1 is a block diagram showing the configuration of a biosensor with exercise amount measuring function according to one embodiment of the present invention.

FIG. 2 is a flowchart showing the operation of a biosensor with exercise amount measuring function according to one embodiment of the present invention.

FIG. 3 is a flowchart showing the operation of a biosensor with exercise amount measuring function according to another embodiment of the present invention.

FIG. 4 is a compared bar graph showing an individual's 30-day calorie-burn values and blood glucose levels measured by the biosensor of the present invention, wherein the rectangle bars represent the blood glucose levels and the pyramid bars represent calorie-burn values.

FIG. 5 is a pie chart showing an individual's living habits measured by the biosensor of the present invention.

FIG. 6 is a chart of 30-day blood glucose management made from figures output by the biosensor of the present invention.

### DETAILED DESCRIPTION OF THE INVENTION

One aspect of the present invention is to provide a biosensor with exercise amount measuring function, comprising: a microprocessor, for processing signals and data; a movement-sensing module connected to the microprocessor, for detecting a movement signal of the user; a biological signal-detecting module connected to the microprocessor, for detecting a biological signal of the user; a flash memory connected to the microprocessor, for storing an exercise amount and biological signal data generated by the microprocessor; and a display unit connected to the microprocessor, for displaying the exercise amount and the biological signal data of the user.

In a preferable embodiment of the present invention, the biological signal-detecting module of the biosensor with exercise amount measuring function of the invention can detect the user's biological signal by reacting a biological sample (such as the user's blood, saliva, cerebro-spinal fluid, tear or perspiration) with chemicals. In a preferable embodiment, the biological signal is a blood glucose value.

In an another embodiment if the invention, the biological signal-detecting module of the biosensor of the present invention further comprises an amplifier, a wave filter and an analog-to-digital converting circuit. The amplifier converts the current into a voltage and amplifies the voltage to match the input requirement of the circuit, and then the wave filter filters out undesired noises, so that the analog-to-digital converting circuit can convert the voltage into processable data and send the data to the microprocessor for further process, thereby obtaining the user's biological signal.

In the biosensor of the present invention, the movement-sensing module can be accomplished by various kinds of applications, and the preferable (but is not limited to) embodiment of the movement-sensing module comprises: a first-direction accelerometer, for sensing the movement signal generated when the user moves in a first direction; a second-direction accelerometer, for sensing the movement signal generated when the user moves in a second direction; and a third-direction accelerometer, for sensing the movement signal generated when the user moves in a third direction. In addition, the movement-sensing module further comprises an amplifier and a wave filter for amplifying movement signals received by the biosensor and filtering out noises from the received movement signals.

As used herein, the term “first-direction accelerometer, second-direction accelerometer and third-direction accelerometer” jointly refer to a “triaxial accelerometer” that detects changes in accelerative force with respect to a three dimensional space to convert movements such as swaying, falling, going up or going down into electrical signals, and determines the corresponding activity pattern by reading the waveform caused by change of velocity. The microprocessor receives the corresponding activity pattern and uses a look-up table to calculate calories burned by the exercise amount. Accumulative calorie-burn of a day can be also calculated based on an accumulative exercise amount. For example, from the waveform variation detected by the accelerometer, a user's activities may be classified into sleeping, sitting, standing and walking, and the microprocessor, after receiving the corresponding activity pattern, calculates the daily calorie-burn using exercise indexes of sleeping, sitting, standing and walking.

The biosensor with exercise amount measuring function of the present invention has a display unit, which may be, for example, a liquid crystal display, for informing the user of the operation process and the testing or measurement results.

The biosensor with exercise amount measuring function of the present invention has the biological signal-detecting module that can further comprise a temperature sensor. Since the biosensor itself and its test strips are subject to specific operational temperature ranges, for minimizing unknown variables during measurement, the biological signal-detecting module is designed to take measurement and save information about biological signals and movement signals together with information about the ambient temperature. The temperature sensor may be realized by a temperature detecting circuit. For example, the temperature sensor may be a voltage divider circuit that contains a current-type temperature sensing component and a series resistor, and is equipped with electronic switches for switching among outputs from a current/voltage converter, an amplifier circuit or itself as the signal source.

The biosensor with exercise amount measuring function of the present invention further comprises a real-time clock, which serves to generate time information and provide information about the duration of the user's use to the biosensor, for the biosensor to display the current time and save the time-related information together with the information about the received corresponding biological signal and movement signal as a record. Such records allow the user to conveniently track and manage his/her individual history of the daily biological signals and movement signals.

Moreover, the disclosed biosensor with exercise amount measuring function further has user interface that preferably include (but is not limited to) four operational keys, namely “+,” “−,” “Menu,” and “Enter.” Of course, the user interface may be provided electronically in a touch screen of the biosensor.

Furthermore, the biosensor with exercise amount measuring function of the present invention may be installed therein with a buzzer or a voice carrier or a chip preloaded with audio files and an audio driver, for providing audio guidance during the user's operating the biosensor.

The inventive biosensor with exercise amount measuring function also has a transmission device for transmitting the user's individual biological signals and daily exercise frequency or calorie-burn information to an external personal computer, so that the computer may use its data transmission function to send such data to a remote medical care system, for medical staff to evaluate or use other software or means to analyze the figures contained in the data, thereby planning more efficient exercise programs for the user.

Another aspect of the invention is to provide a method for measuring an exercise amount using the biosensor of the present invention, comprising: an initialization step, where normal information is inputted; a personalization step, where personal information is inputted; a zeroing step, where an exercise amount is counted from zero; an accelerator-signal recording step, where a movement signal is recorded; a time determining step, where whether it is after 24:00 of a day is determined, and if it is not, the method returns to the accelerator-signal recording step; and if it is, an exercise amount summing step for calculating an accumulated exercise amount of the previous day is performed, and then the method returns to the zeroing step to count the exercise amount of a new day from zero.

A further aspect of the invention is to provide a remote medical system, comprising a network system, a home healthcare system connected to the network system and a medical care system connected to the network system, wherein the home healthcare system uses a biosensor of the present invention to sense movement signals and physiological signals from a user.

Hereinafter, the present invention will be described in more detail with reference to the following embodiments, which are provided by way of example only and should not be construed as limiting the scope thereof. Even so, the examples should not be construed to unduly limit the present invention as modifications and variations in the embodiments discussed herein may be made by those having ordinary skill in the art without departing from the spirit or scope of the present inventive discovery.

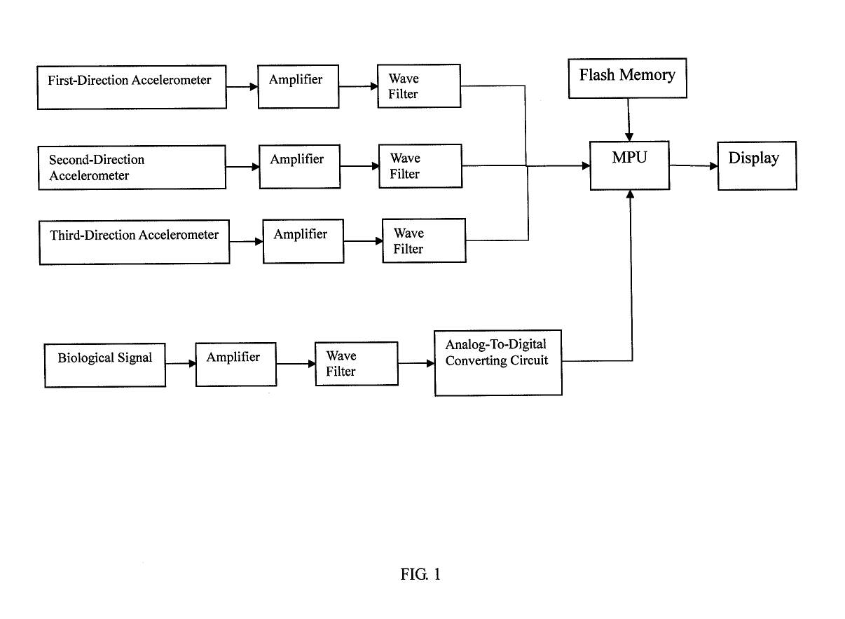

Please first refer to FIG. 1 for a block diagram of a biosensor with exercise amount measuring function according to one embodiment of the present invention. As depicted, the biosensor comprises a movement-sensing module, which is connected to a microprocessor. The movement-sensing module comprises a first-direction accelerometer, for sensing the movement signal generated when the user moves in a first direction; a second-direction accelerometer, for sensing the movement signal generated when the user moves in a second direction; and a third-direction accelerometer, for sensing the movement signal generated when the user moves in a third direction. In addition, the movement-sensing module further comprises an amplifier and a wave filter for amplifying movement signals received by the biosensor and filtering out noises from the received movement signals. The biosensor further comprises a biological signal-detecting module that includes an amplifier, a wave filter and an analog-to-digital converting circuit. The signals received by the two modules are transmitted to and processed by the microprocessor, so as to be displayed in a display unit of the biosensor.

In the biological signal-detecting module of the biosensor, when a user's body fluid reacts with certain chemicals and current variation is therefore generated, the amplifier converts the current into a voltage and amplifies the voltage to match the input requirement of the circuit, and then the wave filter filters out undesired noises, so that the analog-to-digital converting circuit can convert the voltage into processable data and send the data to the microprocessor for further process, thereby obtaining the user's blood glucose level.

The following description will be directed to an exercise module and how an individual daily exercise amount can be determined according thereto.

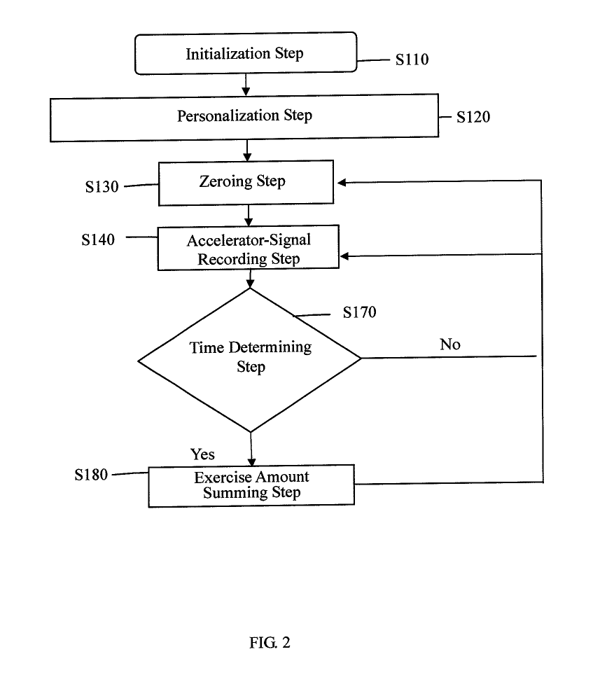

In the present embodiment, the exercise amount can be obtained by measuring how many times the exercise module vibrate (or the number of the user's steps) as a base for calculating the individual daily calorie-burn. FIG. 2 is a flowchart showing the operation of a biosensor with exercise amount measuring function according to one embodiment of the present invention. In an initialization step S110 (of course, when used to measure the exercise amount, the biosensor has to be worn by a user, and the biosensor may have a wrist-wearing design for being worn around the user's wrist or may have an ankle-wearing design for being worn around the user's ankle), the user may input normal data, such as the current year, date and time, and set the desired units of measurement. Afterward, in a personalization step S120, the user may input personal data, such as his/her age, gender, body height and weight. The disclosed biosensor may be equipped with a voice carrier so as to provide audio guidance throughout the user's operation. After the user follows the audio guidance to enter his/her personal data, such as his/her age, gender, body height and weight, a zeroing step S130 is performed to zero any previously stored exercise amount, thereby ensuring the exercise amount will be counted from zero. Then, in an accelerator-signal recording step S140, a vibration frequency caused by the user's exercise (or the user's steps) is counted. Later, in a time determining step S170, whether it is after 24:00 of a day is determined, and if it is not, the method returns to the accelerator-signal recording step S140; and if it is, an exercise amount summing step S180 for calculating an accumulated exercise amount of the previous day is performed, and then the method returns to the zeroing step S130 to count the exercise amount of a new day from zero.

Table 1 below shows figures related to everyday vibration times and everyday blood glucose levels for a time period of one month output by the biosensor of the present invention.

TABLE 1

| | Date | | | | | | | | | |
|---|---|---|---|---|---|---|---|---|---|---|
| | 2010 | 2010 | 2010 | 2010 | 2010 | 2010 | 2010 | 2010 | 2010 | 2010 |
| | Oct. 01 | Oct. 02 | Oct. 03 | Oct. 04 | Oct. 05 | Oct. 06 | Oct. 07 | Oct. 08 | Oct. 09 | Oct. 10 |
| High value of | 264 | 270 | 256 | 234 | 285 | 379 | 400 | 375 | 364 | 325 |
| blood sugar | | | | | | | | | | |
| Low value of | 185 | 240 | 241 | 233 | 255 | 590 | 375 | 361 | 311 | 251 |
| blood sugar | | | | | | | | | | |
| Mean value | 224.5 | 255 | 248.5 | 233.5 | 270 | 334.5 | 387.5 | 368 | 337.5 | 288 |
| Vibration | 4002 | 3715 | 3864 | 3900 | 3512 | 1908 | 2548 | 4581 | 1897 | 3678 |
| number | | | | | | | | | | |
| | Date | | | | | | | | | |
| | 2010 | 2010 | 2010 | 2010 | 2010 | 2010 | 2010 | 2010 | 2010 | 2010 |
| | Oct. 11 | Oct. 12 | Oct. 13 | Oct. 14 | Oct. 15 | Oct. 16 | Oct. 17 | Oct. 18 | Oct. 19 | Oct. 20 |
| High value of | 311 | 274 | 265 | 287 | 310 | 351 | 420 | 467 | 374 | 258 |
| blood sugar | | | | | | | | | | |
| Low value of | 200 | 263 | 241 | 186 | 184 | 311 | 390 | 450 | 310 | 255 |
| blood sugar | | | | | | | | | | |
| Mean value | 255.5 | 268.5 | 253 | 236.5 | 247 | 331 | 405 | 458.5 | 342 | 234.5 |
| Vibration | 3341 | 3289 | 3748 | 3812 | 3895 | 1955 | 3521 | 3341 | 1876 | 3964 |
| number | | | | | | | | | | |
| | Date | | | | | | | | | |
| | 2010 | 2010 | 2010 | 2010 | 2010 | 2010 | 2010 | 2010 | 2010 | 2010 |
| | Oct. 21 | Oct. 22 | Oct. 23 | Oct. 24 | Oct. 25 | Oct. 26 | Oct. 27 | Oct. 28 | Oct. 29 | Oct. 30 |
| High value of | 270 | 300 | 385 | 367 | 347 | 395 | 361 | 347 | 333 | 311 |
| blood sugar | | | | | | | | | | |
| Low value of | 268 | 230 | 260 | 251 | 344 | 364 | 345 | 241 | 218 | 290 |
| blood sugar | | | | | | | | | | |
| Mean value | 269 | 265 | 322.5 | 309 | 345.5 | 379.5 | 353 | 294 | 275.5 | 300.5 |
| Vibration | 3648 | 3612 | 1879 | 2016 | 1840 | 1411 | 1278 | 3261 | 3567 | 3198 |
| number | | | | | | | | | | |

Note:
unit of blood sugar is mg/dl

In the way as described previously, the microprocessor can calculate the individual daily activity level from the movement signals sensed by the accelerometer.

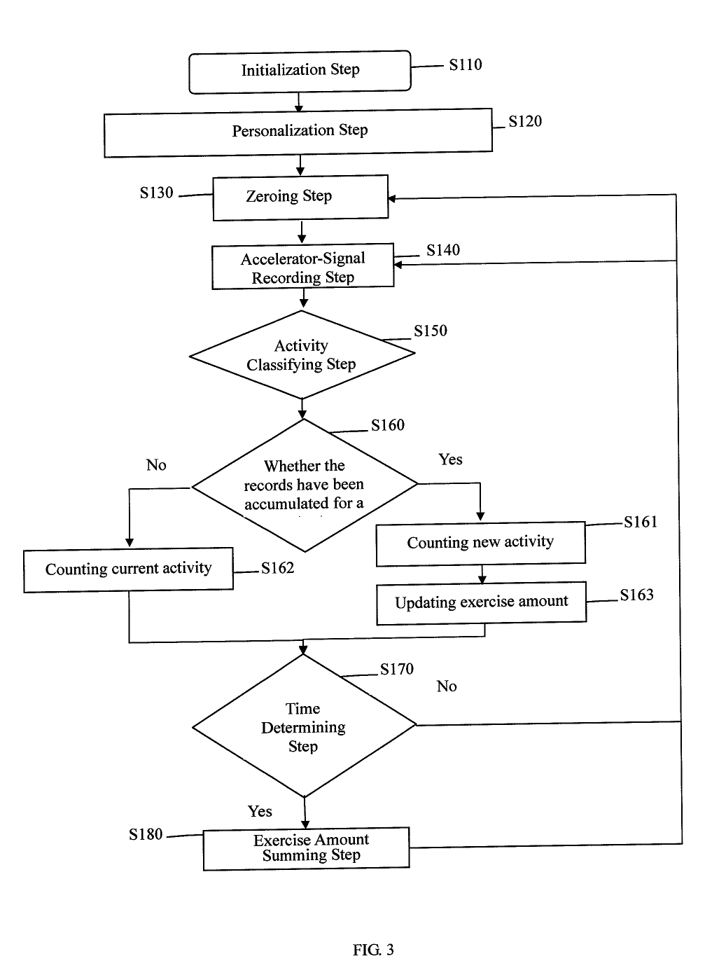

In another embodiment, the exercise amount calculated from the measured vibration times of the exercise module or the measured steps of the user is converted into a calorie-burn value, so as to know the user's daily calorie-burn. FIG. 3 is a flowchart showing the operation of a biosensor with exercise amount measuring function according to another embodiment of the present invention. Different from the foregoing embodiment, the operation of the present embodiment further includes an activity determining step performed between the accelerator-signal recording step S140 and the time determining step S170. The activity determining step includes an activity classifying step S150 for determining the user's current status and a step for determining whether the records have been accumulated for a certain time period S160. In the latter step S160, if the determination is positive, the process proceeds to a step for counting the exercise amount of a new activity S161 and a step for updating the exercise amount S163. If the determination is negative, the process proceeds to a step for counting the exercise amount of the current activity S162.

In this embodiment, the user's activities are classified by the triaxial accelerometer into, for example, sleeping, sitting, standing and walking. The following description will be directed to the different operational patterns of the accelerometer in response to different activities. When the accelerometer detects that the acceleration waveform has remained unchanged for a predetermined time period (for example, 10 minutes), the user's current activity is determined as sleeping because the user stays still. When the user sits, the triaxial accelerometer will only exhibit changes in one dimension. Particularly, when the acceleration waveform first reflects a downward acceleration and then an upward acceleration, it is confirmed that the user is now standing. On the contrary, when the acceleration waveform first reflects an upward acceleration and then a downward acceleration waveform, it is confirmed that the user is now sitting. When the user is walking, his/her hands, feet and trunk are all moving, vibrations happen in all of the three directions. After the vibrations continue for a predetermined time period, the user's current activity is determined as walking.

In addition, in the present embodiment, the exercise amount is converted into the value of burned calories. One exemplificative conversion formula for an individual's daily calorie-burn is:

Physical Activity=BMR×Physical Activity Index

where BMR (Basal Metabolic Rate) is the minimum calorie requirement needed to run an individual's vitals. Table 2 below partially shows the 5^{th} edition of “Recommended Daily Nutrient Allowances” issued by the Department of Health, Executive Yuan, R.O.C. (TAIWAN) in 1993.

TABLE 2
BMR of each Aging distribution

| Year | Male [Kcal/Kg/min] | Female [Kcal/Kg/min] |
|---|---|---|
| 7-9 | 0.0295 | 0.0279 |
| 10-12 | 0.0244 | 0.0231 |
| 13-15 | 0.0205 | 0.0194 |
| 16-19 | 0.0183 | 0.0168 |
| 20-24 | 0.0167 | 0.0162 |
| 25-34 | 0.0159 | 0.0153 |
| 35-54 | 0.0154 | 0.0147 |
| 55-69 | 0.0151 | 0.0144 |
| above 70 | 0.0145 | 0.0144 |

An individual's daily physical activities may be conventionally classified into four levels, namely the sedentary intensity, the light intensity, the moderate intensity and the vigorous intensity, each corresponding to different physical activity indexes. An individual's daily physical activity amount is equal to BMR×Physical Activity Index. Table 3 below discloses the physical activity indexes for activities of the sedentary to light intensity (excluding less-than-one-hour walking or standing during commute, light handiwork or housekeeping, most activity are done sitting down).

TABLE 3
Activity Indexes of Sedentary-to-Light Activities

| Life Activity | Time (hour) | BMR Factor |
|---|---|---|
| Sleeping | 8 | −0.1 |
| Sitting | 12 | +0.5 |
| Standing | 3 | +0.6 |
| Walking | 1 | +1.9 |

Using the foregoing calculation, the microprocessor may refer to a preloaded look-up table that contains information about genders, ages and activities to convert the movement signals sensed by the accelerometer into an everyday calorie-burn value.

A user may, before and/or after taking meals and/or exercise, use a blood glucose test strip and the biosensor of the present invention to measure his/her blood glucose level, sort the measurements by date, and store the measurements in a flash memory connected to the microprocessor.

Table 4 below contains figures output by the disclosed biosensor in an example wherein a 70-year-old, 70-kg male used the biosensor of the present invention to measure and record everyday calorie-burn values and blood glucose levels for a month. Taking Jan. 10, 2010 for example, the user's calorie-burn value of that day is 2265.48 kcal, obtained by:

PA=(0.9×8+1.5×8+1.6×4+2.9×4)×0.0145×60×70=2265.48 (kcal).

TABLE 4

| | Date | | | | | | | | | |
|---|---|---|---|---|---|---|---|---|---|---|
| | 2010 | 2010 | 2010 | 2010 | 2010 | 2010 | 2010 | 2010 | 2010 | 2010 |
| | Oct. 01 | Oct. 02 | Oct. 03 | Oct. 04 | Oct. 05 | Oct. 06 | Oct. 07 | Oct. 08 | Oct. 09 | Oct. 10 |
| High value of | 264 | 270 | 256 | 234 | 285 | 379 | 400 | 375 | 364 | 325 |
| blood sugar | | | | | | | | | | |
| Low value of | 185 | 240 | 241 | 233 | 255 | 590 | 375 | 361 | 311 | 251 |
| blood sugar | | | | | | | | | | |
| Mean value | 224.5 | 255 | 248.5 | 233.5 | 270 | 334.5 | 387.5 | 368 | 337.5 | 288 |

Activity Index I

| | | | | | | | | | | |
|---|---|---|---|---|---|---|---|---|---|---|
| Sleeping (hr) | 8 | 8 | 9.5 | 8 | 8 | 8 | 9 | 8 | 9 | 7.8 |
| Sitting (hr) | 8 | 9 | 9 | 10 | 8.5 | 9 | 9.5 | 12 | 7.5 | 9.2 |
| Standing (hr) | 4 | 4 | 1 | 3 | 4.5 | 3 | 3.5 | 2 | 4 | 3 |
| Walking (hr) | 4 | 3 | 4.5 | 3 | 3 | 4 | 2 | 2 | 3.5 | 4 |
| Calorie (Kcal) | 2265.48 | 2180.22 | 2235.03 | 2174.13 | 2183.265 | 2259.39 | 2055.375 | 2082.78 | 2186.31 | 2266.698 |

| | Date | | | | | | | | | |
|---|---|---|---|---|---|---|---|---|---|---|
| | 2010 | 2010 | 2010 | 2010 | 2010 | 2010 | 2010 | 2010 | 2010 | 2010 |
| | Oct. 11 | Oct. 12 | Oct. 13 | Oct. 14 | Oct. 15 | Oct. 16 | Oct. 17 | Oct. 18 | Oct. 19 | Oct. 20 |
| High value of | 311 | 274 | 265 | 287 | 310 | 351 | 420 | 467 | 374 | 258 |
| blood sugar | | | | | | | | | | |
| Low value of | 200 | 263 | 241 | 186 | 184 | 311 | 390 | 450 | 310 | 255 |
| blood sugar | | | | | | | | | | |
| Mean value | 255.5 | 268.5 | 253 | 236.5 | 247 | 331 | 405 | 458.5 | 342 | 234.5 |

Activity Index I

| | | | | | | | | | | |
|---|---|---|---|---|---|---|---|---|---|---|
| Sleeping (hr) | 10 | 8 | 8 | 8 | 8 | 8 | 8.5 | 8 | 8 | 8 |
| Sitting (hr) | 8 | 9 | 11 | 10 | 11.5 | 11.5 | 11 | 9 | 11 | 12.5 |
| Standing (hr) | 3 | 4.5 | 2 | 2 | 2 | 1.5 | 1.5 | 3 | 2 | 2.5 |
| Walking (hr) | 3 | 2.5 | 3 | 4 | 2.5 | 3 | 3 | 4 | 3 | 1 |
| Calorie (Kcal) | 2101.05 | 2140.635 | 2168.01 | 2253.3 | 2125.41 | 2164.995 | 2146.725 | 2259.39 | 2168.01 | 2000.565 |

| | Date | | | | | | | | | |
|---|---|---|---|---|---|---|---|---|---|---|
| | 2010 | 2010 | 2010 | 2010 | 2010 | 2010 | 2010 | 2010 | 2010 | 2010 |
| | Oct. 21 | Oct. 22 | Oct. 23 | Oct. 24 | Oct. 25 | Oct. 26 | Oct. 27 | Oct. 28 | Oct. 29 | Oct. 30 |
| High value of | 270 | 300 | 385 | 367 | 347 | 395 | 361 | 347 | 333 | 311 |
| blood sugar | | | | | | | | | | |
| Low value of | 268 | 230 | 260 | 251 | 344 | 364 | 345 | 241 | 218 | 290 |
| blood sugar | | | | | | | | | | |
| Mean value | 269 | 265 | 322.5 | 309 | 345.5 | 379.5 | 353 | 294 | 275.5 | 300.5 |

Activity Index I

| | | | | | | | | | | |
|---|---|---|---|---|---|---|---|---|---|---|
| Sleeping (hr) | 8 | 9 | 8 | 7.5 | 8 | 10 | 8 | 7 | 8 | 8 |
| Sitting (hr) | 11 | 9 | 13 | 11.5 | 10 | 8 | 8.5 | 11 | 13 | 11.5 |
| Standing (hr) | 2.5 | 3 | 1 | 1 | 3.5 | 3 | 4 | 4 | 2 | 2.5 |
| Walking (hr) | 2.5 | 3 | 2 | 4 | 2.5 | 3 | 3.5 | 2 | 1 | 2 |
| Calorie (Kcal) | 2128.455 | 2137.59 | 2076.69 | 2265.48 | 2134.545 | 2101.05 | 2222.85 | 2131.5 | 1997.52 | 2085.825 |

Note:
unit of blood sugar is mg/dl

In addition, another purpose of the present invention Is o provide a remote medical system, comprising a network system, a home healthcare system connected to the network system and a medical care system connected to the network system, wherein the home healthcare system uses the biosensor with exercise amount measuring function of the present invention to sense movement signals and physiological signals from a user.

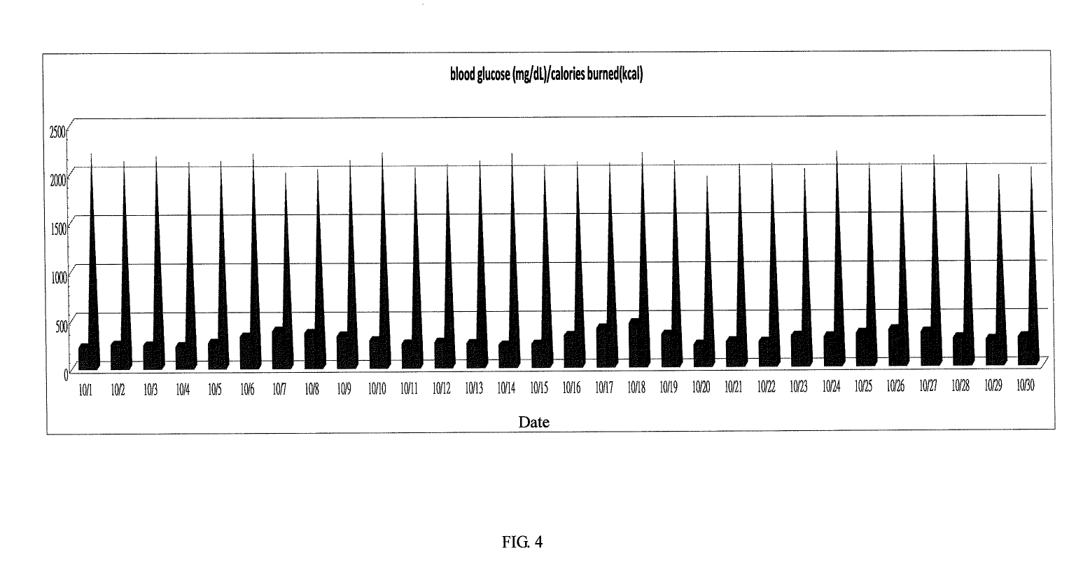

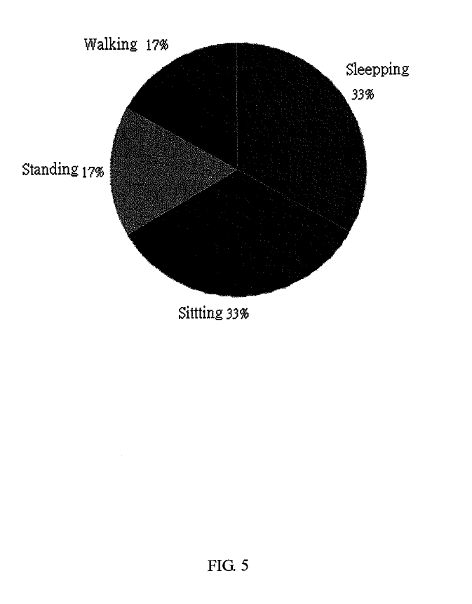

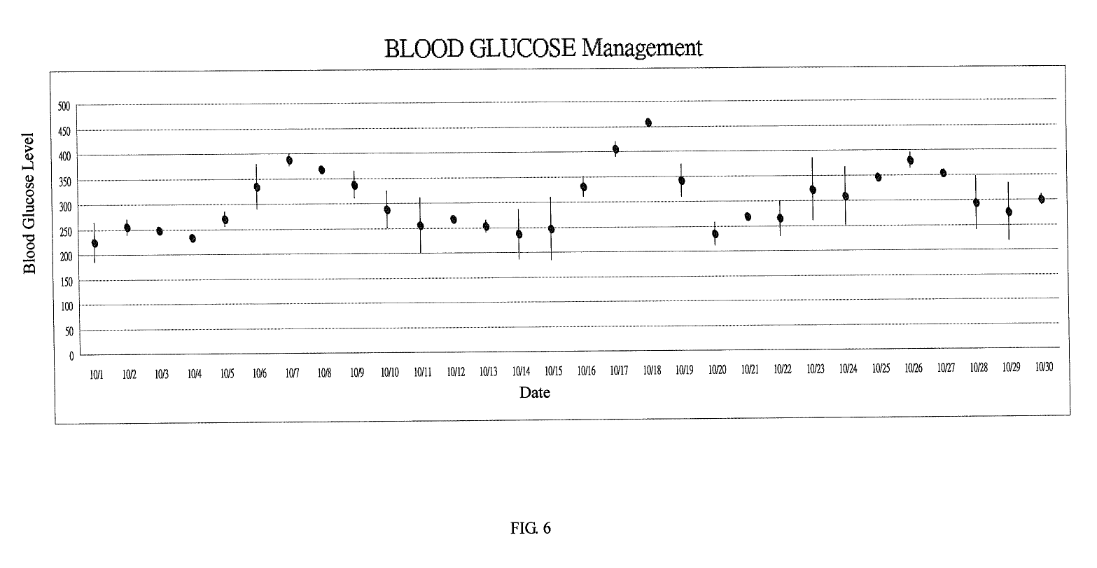

The biosensor with exercise amount measuring function of the present invention further has a transmission device through which the measured individual biological signals and calorie-burn values can be transmitted to a communicated personal computer, and further sent to a medical care system in a hospital through a network system, so that medical staff in the hospital can evaluate or use other software applications to analyze the data, thereby understanding the full profile of the biological signals and calorie-burn values, and accordingly planning more efficient exercise programs for the user. Furthermore, the figures output by the biosensor can be imported to applicable software applications and output as a bar diagram showing everyday blood glucose levels and calorie-burn values graphically, as shown in FIG. 4, or as output as a pie chart summarizing the user's daily activities, as shown in FIG. 5, for medical staff to use as a reference and accordingly suggest adjustment to the user's living habits. The data may be output and/or displayed in various modes as desired by the user. For example, in the event that only the blood glucose levels are needed, they can be extracted independent of the measured exercise amounts and output as the chart of blood glucose management shown in FIG. 6. Of course, the exercise amounts can be extracted independently. Also, the transmission device may be a USB connecting device or an infrared transmitter, or alternatively by bluetooth technology transmitted by means of radio frequency transmission or wireless communication, so as to transmit the data stored in the biosensor to an external computer.

While the invention has been described by means of specific embodiments, numerous modifications and variations could be made thereto by those skilled in the art without departing from the scope and spirit of the invention set forth in the claims.

What is claimed is:

1. A biosensor with exercise amount measuring function, comprising:
a microprocessor, for processing signals and data;
a movement-sensing module connected to the microprocessor, for detecting a movement signal of the user;
a biological signal-detecting module connected to the microprocessor, for detecting a biological signal of the user;
a flash memory connected to the microprocessor, for storing an exercise amount and biological signal data generated by the microprocessor; and
a display unit connected to the microprocessor, for displaying the exercise amount and the biological signal data of the user.

2. The biosensor of claim 1, wherein the biological signal is a blood glucose value.

3. The biosensor of claim 1, wherein the movement-sensing module comprises:
a first-direction accelerometer, for sensing the movement signal generated when the user moves in a first direction;
a second-direction accelerometer, for sensing the movement signal generated when the user moves in a second direction; and
a third-direction accelerometer, for sensing the movement signal generated when the user moves in a third direction.

4. The biosensor of claim 3, wherein the movement-sensing module classifies different activities of the user from a waveform outputted by the accelerometer.

5. The biosensor of claim 4, wherein the activities comprise sleeping, sitting, standing and walking.

6. The biosensor of claim 5, wherein the microprocessor converts the movement signal into a daily calorie-burn value.

7. The biosensor of claim 1, wherein the movement-sensing module further comprises an amplifier, a wave filter and an analog-to-digital converting circuit; and the biological signal-detecting module comprises an amplifier, a wave filter and an analog-to-digital converting circuit.

8. The biosensor of claim 1, further comprising a transmission device.

9. The biosensor of claim 8, wherein the transmission device is a USB connecting device, an infrared transmitter or a bluetooth technology transmitted by means of radio frequency transmission or wireless communication.

10. A method for measuring an exercise amount using the biosensor of claim 1, comprising:
an initialization step, where normal information is inputted;
a personalization step, where personal information is inputted;
a zeroing step, where an exercise amount is counted from zero;
an accelerator-signal recording step, where a movement signal is recorded;
a time determining step, where whether it is after 24:00 of a day is determined, and if it is not, the method returns to the accelerator-signal recording step; and if it is, an exercise amount summing step for calculating an accumulated exercise amount of the previous day is performed, and then the method returns to the zeroing step to count the exercise amount of a new day from zero.

11. The method of claim 10, further comprising an activity determining step performed between the accelerator-signal recording step and the time determining step, wherein the activity determining step comprises an activity classifying step for determining the user's current status and a step for determining whether records have been accumulated for a certain time period, in which if the records have been accumulated for a certain time period, the method proceeds to a step for counting the exercise amount of a new activity and a step for updating the exercise amount, if the records have not been accumulated for a certain time period, the method proceeds to a step for counting the exercise amount of a current activity.

12. The method of claim 11, wherein the activity is classified into four categories as sleeping, sitting, standing or walking by a triaxial accelerometer.

13. A remote medical system, comprising a network system, a home healthcare system connected to the network system and a medical care system connected to the network system, wherein the home healthcare system uses a biosensor with exercise amount measuring function to sense movement signals and physiological signals from a user, and the biosensor comprises:
a microprocessor, for processing signals and data;
a movement-sensing module connected to the microprocessor, for detecting a movement signal of the user;
a biological signal-detecting module connected to the microprocessor, for detecting a biological signal of the user;
a flash memory connected to the microprocessor, for storing an exercise amount and biological signal data generated by the microprocessor; and
a display unit connected to the microprocessor, for displaying the exercise amount and the biological signal data of the user.

14. The remote medical system of claim 13, wherein the biological signal is a blood glucose value.

15. The remote medical system of claim 13, wherein the movement-sensing module comprises:
a first-direction accelerometer, for sensing the movement signal generated when the user moves in a first direction;
a second-direction accelerometer, for sensing the movement signal generated when the user moves in a second direction; and
a third-direction accelerometer, for sensing the movement signal generated when the user moves in a third direction.

16. The remote medical system of claim 15, wherein the movement-sensing module classifies different activities of the user from a waveform outputted by the accelerometer.

17. The remote medical system of claim 16, wherein the activities comprise sleeping, sitting, standing and walking.

18. The remote medical system of claim 17, wherein the microprocessor converts the movement signal into a daily calorie-burn value.

19. The remote medical system of claim 13, wherein the movement-sensing module further comprises an amplifier, a wave filter and an analog-to-digital converting circuit; and the biological signal-detecting module comprises an amplifier, a wave filter and an analog-to-digital converting circuit.

20. The remote medical system of claim 13, wherein the biosensor with exercise amount measuring function further comprises a transmission device.

21. The remote medical system of claim 20, wherein the transmission device is a USB connecting device, an infrared transmitter or a bluetooth technology transmitted by means of radio frequency transmission or wireless communication.
